(19) United States
(12) Patent Application Publication
(10) Pub. No.: US 2026/0151545 A1
(43) Pub. Date: Jun. 4, 2026

(54) REVERSIBLE RADIOPAQUE OCCLUSIVE COMPOSITIONS AND METHODS FOR IMPLANTING THEM

(71) Applicant: Contraline, Inc., Charlottesville, VA (US)

(72) Inventors: Yelena Tropsha, Plymouth, MN (US); Nicholas Matsumoto, Charlottesville, VA (US); Tyler Chiartas, Charlottesville, VA (US); Kevin S. Eisenfrats, Charlottesville, VA (US)

(21) Appl. No.: 19/122,856

(22) PCT Filed: Oct. 20, 2023
(86) PCT No.: PCT/US2023/077473

§ 371 Date: Apr. 21, 2025

Related U.S. Application Data
(60) Provisional application No. 63/417,753, filed on Oct. 20, 2022.

(51) Int. Cl. A61L 31/18; A61L 31/06

(52) U.S. Cl. CPC A61L 31/18; A61L 31/06

(57) ABSTRACT

Compositions, devices, and methods of using one or more hydrogel for contraception are disclosed. In embodiments, the compositions and devices comprise one or more contrast agent, rendering the compositions and devices radiopaque. Included are compositions for an occlusive implant comprising: a multi-arm polyethylene glycol terminated with a thiol crosslinked with a multi-arm polyethylene glycol terminated with a maleimide; and one or more contrast agent. The devices/hydrogel may be used for occlusion of a bodily duct, such as the vas deferens and/or fallopian tubes, for male and female contraception, respectively.

### CROSS-REFERENCE TO RELATED APPLICATIONS

This application relies on the disclosure of and claims priority to and the benefit of the filing date of U.S. Provisional Application No. 63/417,753 filed Oct. 20, 2022, which is hereby incorporated by reference herein in its entirety.

### BACKGROUND OF THE INVENTION

### Field of the Invention

The present invention relates to the field of contraception. Provided are compositions for and methods of using one or more radiopaque hydrogel for contraception.

### SUMMARY OF THE INVENTION

Compositions, devices, and methods of using one or more hydrogel for contraception are disclosed. In embodiments, the compositions and devices comprise one or more contrast agent, rendering the compositions and devices radiopaque. Included are compositions for an occlusive implant comprising: a multi-arm polyethylene glycol terminated with a thiol crosslinked with a multi-arm polyethylene glycol terminated with a maleimide; and one or more contrast agent. The devices/hydrogel may be used for occlusion of a bodily duct, such as the vas deferens and/or fallopian tubes, for male and female contraception, respectively.

Radiopaque occlusive compositions of embodiments of the invention include a composition of Aspect 1 comprising: a first component comprising a first crosslinkable functional group; a second component comprising a second crosslinkable functional group; one or more contrast agent; and one or more solvent; wherein the first component, the second component, the one or more contrast agent, and the one or more solvent are capable of forming an end composition that is capable of forming an occlusion within a body; and wherein the one or more contrast agent is present in the end composition at a concentration which provides or enhances radiopacity of the occlusion.

Aspect 2 is a composition for an occlusive implant comprising: a multi-arm polyethylene glycol terminated with a thiol crosslinked with a multi-arm polyethylene glycol terminated with a maleimide; and one or more contrast agent.

Aspect 3 is a composition comprising: a first component comprising polyethylene glycol terminated with a first crosslinkable functional group; and a second component comprising polyethylene glycol terminated with a second crosslinkable functional group; wherein the first component and/or the second component further comprises one or more contrast agent; wherein the first component and the second component, upon mixing, are capable of undergoing a bioorthogonal reaction to form a covalently cross-linked hydrogel as an end composition.

Aspect 4 is the composition of any of Aspects 1-3, wherein the composition, such as the end composition, comprises a covalently crosslinked polymer hydrogel comprising pores.

Aspect 5 is the composition of any of Aspects 1-4, wherein the composition, such as the end composition, comprises a covalently crosslinked polymer hydrogel comprising pores, wherein the pores have a diameter of no more than about 3 microns.

Aspect 6 is the composition of any of Aspects 1-5, wherein the contrast agent is iohexol.

Aspect 7 is the composition of any of Aspects 1-6, wherein: the first component and the second component comprise polyethylene glycol.

Aspect 8 is the composition of any of Aspects 1-7, wherein: the first crosslinkable functional group is a thiol.

Aspect 9 is the composition of any of Aspects 1-8, wherein: the second crosslinkable functional group is a maleimide.

Aspect 10 is the composition of any of Aspects 1-9, wherein the first component and/or the second component are present in the composition at a concentration of up to about 30 wt %.

Aspect 11 is the composition of any of Aspects 1-10, wherein the first component and/or the second component are present in the composition at up to about 20 wt %.

Aspect 12 is the composition of any of Aspects 1-11, wherein the first component and the second component are present in the composition in equal amounts.

Aspect 13 is the composition of any of Aspects 1-12, wherein the first component and the second component are present in the composition in unequal amounts.

Aspect 14 is the composition of any of Aspects 1-13, wherein the solvent is a buffer comprising citric acid.

Aspect 15 is the composition of any of Aspects 1-14, wherein the first functional group is a thiol.

Aspect 16 is the composition of any of Aspects 1-15, wherein the second functional group is a maleimide.

Aspect 17 is the composition of any of Aspects 1-16, wherein the first component and the second component are dissolved in one or more solvents.

Aspect 18 is the composition of any of Aspects 1-17, wherein the first component and/or the second component has a molecular weight of up to about 50 kDa.

Aspect 19 is the composition of any of Aspects 1-18, wherein the polyethylene glycol of the first component and/or the second component comprises 4 arms.

Aspect 20 is the composition of any of Aspects 1-19, wherein the composition has a gelation rate of less than 30 seconds.

Aspect 21 is the composition of any of Aspects 1-20, comprising a multi-arm polyethylene glycol terminated with a thiol and having a weight percent ranging from about 1 to 30% in the composition and/or a multi-arm polyethylene glycol terminated with a maleimide and having a weight percent ranging from about 1 to 30% in the composition.

Aspect 22 is the composition of any of Aspects 1-21, comprising a multi-arm polyethylene glycol terminated with a thiol and/or a multi-arm polyethylene glycol terminated with a maleimide that is Y-shaped, 3-arm, 4-arm, 6-arm, or 8 arm.

Aspect 23 is the composition of any of Aspects 1-22, wherein the composition is in a form capable of being extruded from a needle.

Aspect 24 is a method of providing contraception, the method comprising injecting the composition of any of Aspects 1-23 into a fallopian tube or vas deferens.

Aspect 25 is a method of implanting the composition of any of Aspects 1-23 the method comprising: injecting the first component and the contrast agent into a target region of a body; injecting the second component into the body; wherein the injecting is optionally performed under imaging guidance; and allowing the first component, the contrast agent and second component to form a mass.

Aspect 26 is a method of implanting the composition of any of Aspects 1-23, the method comprising: identifying a target region within a body; injecting a selected volume of a test composition into the target region; visualizing the target region and the injected volume of the test composition using imaging; removing the test composition from the target region, optionally flushing the test composition from the target region with one or more flushing liquid; selecting a desired length for an implanted hydrogel based on the visualizing; determining an injection volume of a composition capable of forming the hydrogel that corresponds to the desired length for the implanted hydrogel; and injecting the composition capable of forming the hydrogel into the target region.

Aspect 27 is the method of any of Aspects 24-26, wherein the target region is a vas deferens or fallopian tube.

Aspect 28 is the method of any of Aspects 24-27, wherein the imaging is fluoroscopy.

Aspect 29 is the method of any of Aspects 24-28, wherein the injection volume is less than about 200 microliters.

Aspect 30 is the method of any of Aspects 24-29, wherein the injection volume is less than about 175 microliters.

Aspect 31 is the method of any of Aspects 24-30, wherein the test composition comprises a contrast agent, such as iohexol.

Aspect 32 is the method of any of Aspects 24-31, wherein the flushing liquid comprises saline.

Aspect 33 is the method of Aspect 24, wherein the contraception is temporary or permanent contraception.

Aspect 34 is a kit comprising: a first container containing a first component, the first component being a polyethylene glycol (PEG) based component which terminates with a first crosslinkable functional group; a second container containing a second component, the second component being a polyethylene glycol (PEG) based component which terminates with a second crosslinkable functional group; and a delivery device configured to receive the first container and the second container and to dispense at least a portion of the contents of the first container and/or the second container.

Aspect 35 is the kit of Aspect 34, wherein the first component is a multi-arm polyethylene glycol terminated with a thiol functional group.

Aspect 36 is the kit of Aspects 34 or 35, wherein the second component is a multi-arm polyethylene glycol terminated with a maleimide functional group.

Aspect 37 is the kit of any of Aspects 34-36, wherein the first container and/or the second container contains one or more contrast agent.

### BRIEF DESCRIPTION OF THE DRAWINGS

The accompanying drawings illustrate certain aspects of implementations of the present disclosure, and should not be construed as limiting. Together with the written description the drawings serve to explain certain principles of the disclosure.

FIG. 1 is a fluoroscopy image of various hydrogel formulations comprising contrast agents according to embodiments of the invention.

FIG. 2 is a fluoroscopy image showing a 0.25 mL injection of hydrogel comprising iohexol in the right vas and a 0.25 mL injection of Omnipaque™ contrast agent in the left vas.

FIGS. 3A-C are fluoroscopy images showing vasa injected with a hydrogel formulation comprising iohexol according to an embodiment of the invention.

FIG. 3D is a graph showing an estimated maximum safe implant dose for administration into a vas deferens.

FIG. 4 is a fluoroscopy image comparing 0.25 mL and 0.1 mL injections of a hydrogel into vasa according to an embodiment of the invention.

FIGS. 5A-B are drawings of macromers used to prepare hydrogels according to embodiments of the invention.

FIG. 5C is a graph showing rheology testing results for hydrogels prepared according to embodiments of the invention.

FIGS. 6A-C are graphs showing sperm collection data for a first canine test subject including volume (FIG. 6A), count (FIG. 6B), and motility (FIG. 6C) data.

FIGS. 7A-C are graphs showing sperm collection data for a second canine test subject including volume (FIG. 7A), count (FIG. 7B), and motility (FIG. 7C) data.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

Generally, embodiments of the invention relate to hydrogel compositions comprising one or more imaging agent (e.g., a contrast agent) and methods of administering them to a patient. In embodiments, the contrast agent is iohexol (such as in the form of Omnipaque™). In other embodiments, the contrast agent is an iodine-based contrast agent, such as iodixanol (Visipaque™), iopamidol (Isovue™), ioxilan (Oxilan™), iopromide (Ultravist™), iobitridol (Xenetix™), ioversol, metrizamide (Amipaque™), diatrizoate (Hypaque™ or Gastrografin™), metrizoate (Isopaque™), iothalamate (Conray™), or ioxaglate (Hexabrix™). In embodiments, the contrast agent is a gadolinium-based contrast agent, such as gadodiamide (Omniscan™), gadoversetamide (OptiMARK™), gadoxentate (Eovist™), gadobenate (MultiHance™), gadopentetate (Magnevist™), gadoteridol (ProHance™), gadobutrol (Gadavist™), or gadoterate (Clariscan™).

In embodiments, the contrast agent is present in the hydrogel composition at up to about 50 weight percent (wt %), such as about 5, 10, 15, 20, 25, 30, 35, 40, or 45 wt %. The contrast agent may be added to the hydrogel composition as a solid or liquid. In embodiments, the contrast agent may diffuse out of the composition over time, which can be controlled for periods of time ranging from minutes to months.

The hydrogel may comprise one or more additional component. As used herein, the term “component” includes any substance that is capable of forming a hydrogel according to the invention, such as a biomaterial product. For example, a component can include a small molecule, catalyst, peptide, protein, enzyme, nucleotide (or derivatives of), short chains of nucleotides (or derivatives of), long chains of nucleotides (or derivatives of), monosaccharides (or derivatives of), disaccharides (or derivatives of), trisaccharides (or derivatives of), oligo saccharides (or derivatives of), polysaccharides (or derivatives of), monomer, oligomer, macromer, or polymer that can be cross-linked with another component to form a hydrogel according to the invention.

The hydrogel composition or component thereof can include a mixture or solution of one or more constituents (e.g., a polymer and a solvent). A component can include such constituents regardless of their state of matter (e.g., solid, liquid, or gas). A component can include both active constituents and inert constituents. A constituent may be one or more of a therapeutic agent, an active agent, or drug. For example, in some embodiments, a component can include certain polymers that can form a delivered product, as well as a medicament or other active ingredient. By way of another example, in some embodiments a component can include drugs, including but not limited to, small molecule drugs and biologics. In other embodiments, a component can include certain constituents to impart desired properties to the delivered product, including constituents that facilitate the delivered product being echogenic, radiopaque, radiolucent, or the like.

In embodiments, the components (e.g., monomers, macromers, or polymers) that form the hydrogel may have varied molecular weights, component ratios, concentrations/weight percentages of the components in solvent, and composition of the solvent. Varying any, some, or all of these properties can affect the mechanical, chemical, or biological properties of the device. This includes properties such as, but not limited to, dissolution time, gelation rate/time, porosity, biocompatibility, hardness, elasticity, viscosity, swelling, fluid absorbance, melting temperature, degradation rate, density, reversal time, and echogenicity. Accordingly, one of skill in the art based on this disclosure will know how to “tune” the particular desired features of a hydrogel to achieve a particular purpose and/or function for a particular application.

In embodiments, the hydrogel can be formed by having one or more substances/components/constituents cross-link with one or more of each other, such as macromers. In embodiments, the hydrogel can be formed in situ and/or otherwise at the time of insertion/injection and/or thereafter, such as immediately upon combination of components.

The hydrogel or its macromers can include components including, but not limited to, a polymer backbone, stimuli-responsive functional group(s), and functional groups that enable cross-linking. The functional groups that enable cross-linking can be end groups on the macromer(s). The cross-linking of the macromers may be via bioorthogonal chemistry, such as a Click reaction. In one embodiment, a bioorthogonal reaction is utilized because it is highly efficient, has a quick gelation rate, occurs under mild conditions, and does not require a catalyst, but in some cases can be performed with a catalyst, e.g., classic azide/alkyne click is generally performed with metal catalyst(s).

Another type of Click reaction is cycloaddition, which can include a 1,3-dipolar cycloaddition or hetero-Diels-Alder cycloaddition or azide-alkyne cycloaddition, for example. The reaction can be a nucleophilic ring-opening. This includes openings of strained heterocyclic electrophiles including, but not limited to, aziridines, epoxides, cyclic sulfates, aziridinium ions, and episulfonium ions. The reaction can involve carbonyl chemistry of the non-aldol type including, but not limited to, the formation of urcas, thioureas, hydrazones, oxime ethers, amides, and aromatic heterocycles. The reaction can involve carbonyl chemistry of the aldol type. The reaction can also involve forming carbon-carbon multiple bonds, epoxidations, aziridinations, dihydroxylations, sulfenyl halide additions, nitrosyl halide additions, and Michael additions.

Another example of bioorthogonal chemistry is nitrone dipole cycloaddition. Click chemistry can include a norbornene cycloaddition, an oxanobornadiene cycloaddition, a tetrazine ligation, a [4+1] cycloaddition, a tetrazole chemistry, or a quadricyclane ligation. Other end-groups include, but are not limited to, acrylic, cyrene, amino acids, amine, or acetyl. In one aspect, the end groups may enable a reaction between the polymeric device and the cells lining the tube, duct, tissue, or organ that is being occluded. For example, the devices, compositions, hydrogels and methods of the present invention can include any device, composition, method, hydrogel and/or component/constituent thereof disclosed in any one or more of U.S. Patent Application Publication Nos. 2017/0136143, 2017/0136144, 2018/0028715, 2018/0185096, 2019/0038454, 2019/0053790, 2019/0060513, 2020/0352649, 2022/0015742, 2022/0168461, 2022/0175672, U.S. Pat. Nos. 10,155,063, 10,751,124, 11,278,641, and International Patent Application Publication Nos. WO2017/083753, WO2018/139369, WO2019/070632, WO2021/035217, which are each incorporated by reference herein in their entireties.

The macromers or polymers that form hydrogels according to embodiments of the invention may be one or more of natural or synthetic monomers, polymers, copolymers or block copolymers, biocompatible monomers, polymers, copolymers or block copolymers, random copolymers, statistical copolymers, alternating copolymers, polystyrene, neoprene, polyetherether ketone (PEEK), carbon reinforced PEEK, polyphenylene, polyetherketoneketone (PEKK), polyaryletherketone (PAEK), polyphenylsulphone, polysulphone, polyurethane, polyethylene, low-density polyethylene (LDPE), linear low-density polyethylene (LLDPE), high-density polyethylene (HDPE), polypropylene, polyetherketoneetherketoneketone (PEKEKK), nylon, fluoropolymers, polytetrafluorocthylene (PTFE or TEFLON®), TEFLON® TFE (tetrafluoroethylene), polyethylene terephthalate (PET or PETE), TEFLON® FEP (fluorinated ethylene propylene), TEFLON® PFA (perfluoroalkoxy alkane), and/or polymethylpentene (PMP) styrene maleic anhydride, styrene maleic acid (SMA), polyurethane, silicone, polymethyl methacrylate, polyacrylonitrile, poly(carbonate-urethane), poly(vinylacetate), nitrocellulose, cellulose acetate, urethane, urethane/carbonate, polylactic acid, polyacrylamide (PAAM), poly (N-isopropylacrylaminc) (PNIPAM), poly (vinylmethylether), poly (ethylene oxide), poly(ethyl (hydroxyethyl) cellulose), polyoxazoline and any of its derivatives (POx), polylactide (PLA), polyglycolide (PGA), poly(lactide-co-glycolide) PLGA, poly(e-caprolactone), polydiaoxanone, polyanhydride, trimethylene carbonate, poly(β-hydroxybutyrate), poly(g-ethyl glutamate), poly(DTH-iminocarbonate), poly(bisphenol A iminocarbonate), poly(orthoester) (POE), polycyanoacrylate (PCA), polyphosphazene, polyethyleneoxide (PEO), polyethyleneglycol (PEG) or any of its derivatives, linear or multi-armed PEG and any of its derivatives, polyacrylacid (PAA), polyacrylonitrile (PAN), polyvinylacrylate (PVA), polyvinylpyrrolidonc (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), N-isopropylacrylamide (NIPA), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, keratin, pectin, elastin, ethylene vinyl acetate, ethylene vinyl alcohol (EVOH), polyethylene oxide, PLA or PLLA (poly(L-lactide) or pol (L-lactic acid)), poly(D,L-lactic acid), poly(D,L-lactide), polydimethylsiloxane or dimethicone (PDMS), poly(isopropyl acrylate) (PIPA), polyethylene vinyl acetate (PEVA), PEG styrene, polytetrafluorethylene RFE, TEFLON® RFE, KRYTOX® RFE, fluorinated polyethylene (FLPE or NALGENE®, methyl palmitate, temperature responsive polymers, poly(N-isopropylacryl amide) (NIPA), polycarbonate, polyethersulfone, polycaprolactone, polymethyl methacrylate, polyisobutylene, nitrocellulose, medical grade silicone, cellulose acetate, cellulose acetate butyrate, polyacrylonitrile, poly(lacti de-co-caprolactone (PLCL), and/or chitosan; poly (methyl methacrylate), poly(vinyl alcohol), poly (urethanes) poly (ethylene) poly (siloxanes) or silicones, poly (vinyl pyrrolidone), poly(ethylene-co-vinyl acetate), poly (methyl methacrylate), poly(vinyl alcohol), poly (N-vinyl pyrrolidone), poly(acrylic acid), poly (2hydroxy ethyl methacrylate), polyacrylamide, poly (methacrylic glycol), poly(ethylene glycol), polyorthoesters, poly (lactide-co-glycolides) (PLGA), polyactide (PLA), polyanhydride, polyglycolides (PGA); polymers formed from radical polymerization such as polystyrene, poly (acrylic acid), poly (methacrylic acid), poly(ethyl methacrylate), poly(methyl methacrylate), poly(vinyl acetate), poly(ethyleneterepthalate), polyethylene, polypropylene, polybutadiene, polyacrylonitrile, poly(vinyl chloride), poly(vinylidene chloride), poly(vinyl alcohol), polychloroprene, polyisoprene, vinyl fluoride, vinylidene fluoride, trifluoroethylene, poly(methyl-α-chloracrylate), poly(ethylvinyl ketone), polymethacroleine, polyaurylmethacryate, poly(2-hydroxyethylmethamilate), poly(fumaronitrile), polychlorotrifluoroethylene, poly(acrylonitrile), polyacroleine, polyacenaphthylene, and branched polyethylene; natural polymers including silk, rubber, cellulose, alginate, wool, amber, keratin, collagen, starch, DNA, and shellac.

In one embodiment, the molecular weight of the polymers can be varied from around 1 kDa to 5,000,000 kDa. In embodiments, the molecular weight of the polymer is preferred to be from about 10 kDa to 80 kDa, such as about 20 kDa to 60 kDa, 30 kDa to 50 kDa, or 40 kDa to 45 kDa. Polymers of high molecular weight are preferred, which yield small pores in the device and thus, create an effective occlusion. A high molecular weight can also create a more viscous solution and thus, can be more difficult to inject. In other embodiments, the polymers can have a weight average molecular weight (Mw) or number-average molecular weight (Mn) ranging from about 1,000 to 1,000,000 Daltons for example as measured by GPC (gel permeation chromatography) with polystyrene equivalents, mass spectrometry, or other appropriate methods. The term “about” used herein in the context of quantitative measurements means #10%. For example, with a +10% range, a number average molecular weight (Mn) or the weight average molecular weight (Mw) of “about 1,000 Daltons” can mean a molecular weight in the range of 900-1,100 Daltons. In embodiments, the number average molecular weight (Mn) or the weight average molecular weight (Mw) of polymers of the invention can range from about 1,000 to about 1,000,000 Daltons, such as from about 3,000 to about 60,000 Daltons, or from about 20,000 to about 90,000 Daltons, or from about 150,000 to about 900,000 Daltons, or from about 200,000 to about 750,000 Daltons, or from about 250,000 to about 400,000 Daltons, or from about 300,000 to about 800,000 Daltons, and so on. Further, the degree of polymerization of the polymers in embodiments can range from 1 to 10,000, such as from 50 to 500, or from 500 to 5,000, or from 1,000 to 3,000.

In embodiments, the chain length or degree of polymerization (DP) can have an effect on the properties of the polymers. In the context of this specification, the degree of polymerization is the number of repeating units in the polymer molecule. In embodiments, the polymers include from 2 to about 100,000 repeating units. Preferred are polymers which include from about 5 to 10,000 repeating units, such as from about 10 to 8,000, or from about 15 to 7,000, or from about 20 to 6,000, or from about 25 to 4,000, or from about 30 to 3,000, or from about 50 to 1,000, or from about 75 to 500, or from about 80 to 650, or from about 95 to 1,200, or from about 250 to 2,000, or from about 350 to 2,700, or from about 400 to 2,200, or from about 90 to 300, or from about 100 to 200, or from about 40 to 450, or from about 35 to 750, or from about 60 to 1,500, or from about 70 to 2,500, or from about 110 to 3,500, or from about 150 to 2,700, or from about 2,800 to 5,000, and so on.

In one embodiment, the weight percent, or concentration of the components in solution, is varied from around 1% to around 50% of the component in solvent, such as from 1% to 2%, from 2% to 3%, from 3% to 4%, from 4% to 5%, from 5% to 6%, from 6%, to 7%, from 7%, to 8%, from 8% to 9%, from 9% to 10%, and so on. In another embodiment, the weight percent of the macromer is from around 2.5% to around 20% in the solvent, including 6% to around 20%, 7% to around 20%, 8% to around 20%, as so on. The weight percent can affect the mechanical and chemical properties of the polymer, such as increasing or decreasing pore size, viscosity, hardness, clasticity, density, and degradation.

The solvent that the component is dissolved in can be aqueous (water-based) or an organic solvent (e.g. DMSO, PEG, ethanol). The final composition may contain excipients for purposes such as increased solubility or quicker dissolution rate. The pH of the composition in solution can be varied from 4 to 9, such as from 4 to 5, 5 to 6, 6 to 7, 7 to 8, and 8 to 9. The pH of the solution can affect the gelation time and stability of the macromer in solution.

In one embodiment, the gelation rate and time of formation of the polymer device varies. Gelation can occur instantaneously, in less than 1 minute, or within 1-10 minutes. In some embodiments, the gelation time is less than about 60 seconds, for example, less than about 30 seconds, and in some cases may be instantaneous/immediate. In other embodiments, the gelation time is between about 1 second and 60 seconds. The particular components used to form the hydrogel/device/delivered product can be selected such that the gelation time/rate is “tuned” for the particular application. For example, the components/constituents can be selected to provide for faster or slower gelation times as desired.

The hydrogel, composition, polymer device or otherwise referred to as the delivered product can be a biomaterial that is formed from multiple biomaterial components and delivered with any delivery system to target locations. A delivered product can be the implant or structure that is at least partially formed with the system by multiple biomaterial components that react together or assemble into higher order structures via covalent and/or non-covalent bonds, and that is delivered by the system. For example, in certain situations, the delivered product can have a storage modulus (delivered G′) and a loss modulus (delivered G″) when the first component and the second component are conveyed out of a delivery member. The ratio of the delivered G″ to the delivered G′ can between about ⅓ and about 3. In some embodiments, such as for hydrogels, the delivered G′ can be greater than the delivered G″ (i.e., a ratio of the delivered G″ to the delivered G′ is less than 1), thus indicating that the delivered product is a viscoelastic material. In embodiments, G′ can range from 0.1 to 90,000 and G″ can range from 0.001 to 30,000, such as G′ ranging from 0.1 to 80,000 and G″ ranging from 0.001 to 25,000, such as G′ ranging from 0.1 to 70,000 and G″ ranging from 0.001 to 22,000, such as G′ ranging from 0.1 to 75,000 and G″ ranging from 0.001 to 25,000, such as G′ ranging from 0.1 to 60,000 and G″ ranging from 0.001 to 20,000; additionally or alternatively, the ratio of the delivered G″ to the delivered G′ can between about ⅓ and about 3, such as 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3; additionally or alternatively, such as for hydrogels, the delivered G′ can be greater than the delivered G″ (i.e., a ratio of the delivered G″ to the delivered G′ is less than 1, such a 0.01, 0.02, 0.03, 0.04, 0.05, 0.06, 0.07, 0.08, 0.09, or 0.091-0.099), thus indicating that the delivered product is a viscoelastic material; additionally or alternatively G′ can be 0.1-90,000, 10-85,000, 50-55,000, 100-40,000, 500-35,000, 1,000-10,000, or any range in between such as 10-90,000 or 500-85,000, for example; alternatively or in addition G″ can be 0.001-30,000, such as 0.005-25,000, 0.01-20,000, 0.05-10,000, 0.1-1,000, or any range in between such as 0.05-25,000 or 0.001-1,000. In some embodiments, the components can be formulated such that a viscoelastic substance (and not a liquid substance) is conveyed out of the exit opening of the delivery member. In some embodiments, the hydrogel is conveyed out of the exit opening of the delivery member into a body part, organ, duct, cavity/space or lumen to at least partially or fully occlude the body part, organ, duct, cavity/space or lumen. In some embodiments, the body part, organ, duct, cavity/space or lumen is chosen from an artery, vein, capillary, vessel, tissue, intra-organ space, lymphatic vessel, a femoral artery, popliteal artery, coronary and/or carotid artery, esophagus, cavity, nasopharyngeal cavity, car canal, tympanic cavity, sinus, sinuses of the brain, any artery of the arterial system, any vein of the venous system, heart, larynx, trachea, bronchi, stomach, duodenum, ileum, colon, rectum, bladder, kidney, ureter, ejaculatory duct, epididymis, vas deferens, urethra, uterine cavity, vaginal canal, fallopian tube, cervix, duct, bile duct, a hepatic duct, a cystic duct, a pancreatic duct, a parotid duct, organ, a uterus, prostate, organ of the gastrointestinal tract, organ of the circulatory system, organ of the respiratory system, organ of the nervous system, urological organ, subcutaneous space, intramuscular space, or interstitial space.

In one embodiment, the device/hydrogel/delivered product swells upon contact with one or more fluids inside the body. Swelling allows for the device to secure itself or “lock” within the body part, duct, organ, cavity/space or lumen to form a good occlusion. The device can swell greater than 100%, such as 100-200%, 200-300%, 300-400%, and so on. Swelling may also allow for the device to properly secure itself within the body part, duct, organ, cavity/space or lumen.

According to another embodiment, the device includes pores. The pores are typically dispersed throughout the device. The porosity is defined by the properties of the macromers and cross-linking of the macromers. In embodiments, the pore diameter of the formed polymer ranges from 0.001 nm to 3 μm, such as from 0.001 nm to 1 μm. In other embodiments, the pore diameter ranges from 0.01 nm to 100 nm, or from about 1 nm to about 1 μm. In other embodiments, the pore diameter is 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 15, 20, 25, 30, 35, 40, 45, 50, 55, 60, 65, 70, 75, 80, 95, 90, 95, or 100 nm. Specific pore sizes can be targeted to provide an optimum porosity that provides maximum flow of fluid while blocking the flow of sperm cells or ova. In other embodiments, the pores range from 0.1 nm to 2 microns in diameter. In one embodiment, the device is suitable for occlusion of reproductive cells. The pores are less than 3 μm to prevent the flow of sperm. The pores allow for fluid to travel through the hydrogel. The mesh size of the device is small enough to block reproductive cells from traversing through. In one embodiment, a larger pore size may be desire for quicker release of drug from the hydrogel.

In one embodiment, the hydrogel/device/delivered product does not degrade inside the body in that it is permanent, such as a device capable of providing permanent contraception. In another embodiment, the hydrogel/device/delivered product degrades or is capable of degrading in the body, for example, by way of an endogenous stimulus (e.g., hydrolysis). The degradation rate is slow enough that the device remains an effective occlusion inside the body for greater than three months. According to another embodiment, the device degrades upon application of an exogenous stimulus, for example, by photodegradation (e.g., ultraviolet or infrared exposure), acoustic, and/or enzymatic degradation.

In one embodiment, a multi-syringe system is used to inject or implant the polymeric device for occlusion. Each syringe can inject a separate macromer/component/constituent. The system can also contain a component that mixes the macromer/component/constituent solutions before implanting into the body and has multiple channels that prevent the components from mixing. The macromer/component/constituent cross-link in situ to form the hydrogel/device, such as an occlusive device. In another aspect, the cross-linking is complete within the injection device prior to the hydrogel being implanted into the body. The injection speed and injection volume can be controlled, tuned, or automated. A handheld device may be used for performing the injection. The injection device can be single use and disposable, or can be multiuse with a replaceable cartridge container in which the macromer solutions are delivered. In one aspect, mixing and/or dissolution of the drug and macromer solution is conducted within the multi-syringe system.

In some embodiments, a composition includes a first component and a second component that are each formulated to be crosslinked with the other to form a hydrogel. The first component and the second component are formulated to have an initial storage modulus (initial G′) and an initial loss modulus (initial G″) when initially combined such that a ratio of the initial G″ to the initial G′ is between about 5 and about 100. In embodiments, G′>G″ and/or G′/G″>1 and/or G″/G′<1. The first component and the second component are formulated to have a gelation storage modulus (gelation G′) and a gelation loss modulus (gelation G″) at a gelation time after the first component and the second component are combined such that a ratio of the gelation G″ to the gelation G′ is less than about 5, such as less than about 1. In embodiments, the gelation time is less than about 120 seconds. The term “gelation” refers to the transition of the hydrogel components from a soluble polymer of finite branches to a substance with infinitely large molecules. Similarly stated, “gelation” refers to the condition where the gel forms and after the components are combined. Thus, the gelation time refers to the time that it takes for the resulting hydrogel to substantially reach equilibrium.

In some embodiments, the first component is at least one of a polyvinyl alcohol, alginate or modified alginate, chitosan or modified chitosan, polyethyleneimine, carboxymethyl cellulose, and/or polyethylene glycol terminated with a functional group (e.g., amine, thiol, maleimide, azide, activated ester), such as a bioorthogonal functional group. The second component is at least one of a water or buffer, water or buffer with divalent cations such as calcium, a solution of reduced hyaluronic acid, a solution of polystyrene sulfonate, a solution of gelatin, and/or polyethylene glycol terminated with a functional group (e.g., amine, thiol, maleimide, azide, activated ester), such as a bioorthogonal functional group. In some embodiments, polyvinyl alcohol, alginate, chitosan, polyethyleneimine, carboxymethyl cellulose, polyethylene glycol terminated with functional groups, divalent cations, reduced hyaluronic acid, polystyrene sulfonate, or gelatin have a weight percent ranging from about 1% to 30% in solvent, such as about 2% to 25%, 3% to 22%, 4% to 20%, 5% to 18%, 7% to 15%, or 10% to 12%. In some embodiments the polysaccharides may be modified with different functional groups. In some embodiments the polysaccharides and proteins may range in molecular weight from 10,000-1,000,000 grams/mole. In some embodiments, the polyvinyl alcohol, polystyrene sulfonate, polyethyleneimine, and polyethylene glycol may be linear, hyperbranched, Y-shaped, 3-arm, 4-arm, 6-arm, or 8-arm and range in molecular weight from 1,000-1,000,000 grams/mole.

In some embodiments, the dissolving solution for the polymer component(s) may be aqueous buffers, including any one or more of phosphate, citrate, acetate, histidine, lactate, tromethamine, gluconate, aspartate, glutamate, tartrate, succinate, malic acid, fumaric acid, alpha-ketoglutaric, and/or carbonate. Specific solvents/buffers can include: 1) acetic acid and sodium acetate (AA), 2) citric acid and sodium citrate (CP), 3) citric acid and phosphate buffer (CP), and 4) phosphate buffer (PB), or combinations thereof. Non-aqueous solvents include: dimethyl isosorbide, glycofurol 75, PEG 200, diglyme, tetrahydrofurfuryl alcohol, ethanol, acetone, solketal, glycerol formal, dimethyl sulfoxide, propylene glycol, ethyl lactate, N-methyl-2-pyrrolidone, dimethylacetamide, methanol, isopropanol, 1,4-butanediol, ethyl acetate, toluene, acetonitrile, and combinations thereof.

The molarity of the solutions/solvents/buffers can range for example from 0.1 M to 0.15 M to 0.2 M. In some embodiments, the solution can include a 0.2 M citric acid buffer and can be formulated to have a solution pH of between 4.0 and 6.0. In some embodiments, the pH of the solution can be between 4.0 and 5.25, or about 4.0. In other embodiments, the pH of the solution can be about 5.25. In yet other embodiments, the pH of the solution can be between about 4.5 and about 8 such as a pH of about 5-7, or about 4.5-6.

In Table 1, the buffer (first column) for example can include any of Acetic Acid-Sodium Acetate (AA), Citric Acid-Sodium Citrate (CA), Citric Acid (0.2)—Phosphate Buffer (0.1) (CP), or Phosphate Buffer (PB), or combinations thereof. The molarity (M) is provided in the second column, which can be adjusted depending on the embodiment. The pH is provided in the third column, but can be adjusted for any embodiment to have a pH range of about 4-9, such as about 4.5, 4.75, 5, 5.25, 5.5, 5.75, 6, 6.25, 6.5, 6.75, 7, 7.25, 7.5, 8, or 8.5. The molecular weight (in kDa) is provided in the fourth column, but can also be adjusted such that the polymer has a molecular weight within a desired range. The chemistry of the components is provided in the fifth column, and can include any of the listed combinations including any one or more functional groups chosen from Thiol (SH), Maleimide (MAL), Hydrazide (HZ), Isocyanate (IC), Amine (NH), Succinimidyl Glutaraldehyde (SG), Aldehyde (AD), or Epoxide (EP), or combinations thereof. The weight percentage (in solution) is provided in the sixth column and likewise can be adjusted according to particular applications, such as providing a composition comprising a desired polymer with a weight percent of up to 20 wt %, such as from about 1-5 wt %, or from about 2-10 wt %, or from about 3-15 wt %, or from about 10-20 wt %. The seventh column provides information regarding the delivery rate. Methods of delivery were either via a pipette or via an injection device, such as those similar to those described in U.S. patents application Ser. Nos. 16/681,572 (published as U.S. Patent Application Publication No. 2020/0146876 and Ser. No. 16/681,577 (U.S. Patent Application Publication No. 2020/0147301), each entitled “Systems and Methods for Delivering of Biomaterials,” and each filed Nov. 12, 2019, each which is incorporated herein by reference in its entirety. The units of injection rate are microliters per minute (μL/min). The injection rate is in the range of about 100-10,000 L/min, such as about 200, 300, 400, 500, 600, 700, 800, 900, 1000, 1250, 1500, 1750, 2000, 2250, 2500, 2750, 3000, 3500, 4000, 4500, 5000, 5500, 6000, 6500, 7000, 7500, 8000, 8500, 9000, or 9500 μL/min. The gelation time (seconds) is provided in the last column. A gelation time of “Imm.” indicates that gelation occurred immediately after the two components were combined.

TABLE 1
Formulations according to embodiments of the invention

| Buf | M | pH | MW | Chem | Wt % | Rate | Gel time |
|---|---|---|---|---|---|---|---|
| AA | 0.1 | 4.50 | 20 | SH-MAL | 20 | 566 | ~120 |
| AA | 0.1 | 5.00 | 20 | SH-MAL | 20 | 283 | ~120 |
| AA | 0.2 | 4.50 | 20 | SH-MAL | 20 | 5661 | ~120 |
| AA | 0.2 | 4.50 | 20 | SH-MAL | 20 | 566 | ~120 |
| AA | 0.2 | 4.50 | 20 | SH-MAL | 20 | 283 | ~120 |
| AA | 0.2 | 5.00 | 20 | SH-MAL | 20 | 5661 | ~120 |
| AA | 0.2 | 5.00 | 20 | SH-MAL | 20 | 566 | ~120 |
| AA | 0.2 | 5.00 | 20 | SH-MAL | 20 | 283 | ~120 |
| AA | 0.2 | 5.50 | 20 | SH-MAL | 20 | 566 | ~120 |
| AA | 0.2 | 5.50 | 20 | SH-MAL | 20 | 283 | ~120 |
| CA | 0.1 | 4.50 | 20 | SH-MAL | 20 | 5661 | ~120 |
| CA | 0.1 | 4.50 | 20 | SH-MAL | 20 | 566 | ~120 |
| CA | 0.1 | 4.50 | 20 | SH-MAL | 20 | 283 | ~120 |
| CA | 0.1 | 5.00 | 20 | SH-MAL | 20 | 5661 | ~120 |
| CA | 0.1 | 5.00 | 20 | SH-MAL | 20 | 566 | ~120 |
| CA | 0.1 | 5.00 | 20 | SH-MAL | 20 | 283 | ~120 |
| CA | 0.2 | 4.00 | 20 | SH-MAL | 20 | 283 | ~120 |
| CA | 0.2 | 5.25 | 20 | SH-MAL | 20 | 600 | ~30 |
| CA | 0.2 | 5.25 | 20 | SH-MAL | 20 | 566 | ~30 |
| CA | 0.2 | 5.25 | 20 | SH-MAL | 20 | 500 | ~30 |
| CA | 0.2 | 5.25 | 20 | SH-MAL | 20 | 450 | ~30 |
| CA | 0.2 | 5.25 | 20 | SH-MAL | 20 | 400 | ~30 |
| CA | 0.2 | 5.25 | 20 | SH-EMAL | 20 | 400 | ~60 |
| | | | | (ESTER) | | | |
| CA | 0.2 | 5.25 | 20 | SH-MAL | 20 | 350 | ~30 |
| CA | 0.2 | 5.25 | 20 | SH-MAL | 20 | 300 | ~30 |
| CA | 0.2 | 5.25 | 20 | SH-MAL | 20 | 283 | ~30 |
| CA | 0.2 | 5.25 | 20 | SH-MAL | 15 | 400 | ~30 |
| CA | 0.2 | 5.25 | 20 | SH-MAL | 10 | 400 | ~30 |
| CA | 0.2 | 5.25 | 20 | SH-MAL | 5 | 400 | ~60 |
| CA | 0.2 | 5.25 | 20 | SH-MAL | 2.5 | 400 | ~120 |
| CA | 0.2 | 5.25 | 20 | SH-MAL | 1 | 400 | ~120 |
| CA | 0.2 | 6.00 | 20 | SH-MAL | 2.5 | 400 | ~60 |
| CA | 0.2 | 6.00 | 20 | SH-MAL | 1 | 400 | ~60 |
| CP | 0.15 | 5.50 | 20 | SH-MAL | 20 | 5661 | Imm. |
| CP | 0.15 | 5.50 | 20 | SH-MAL | 20 | 566 | Imm. |
| CP | 0.15 | 5.50 | 20 | SH-MAL | 20 | 283 | Imm. |
| CP | 0.15 | 5.75 | 20 | SH-MAL | 20 | 5661 | Imm. |
| CP | 0.15 | 5.75 | 20 | SH-MAL | 20 | 566 | Imm. |
| CP | 0.15 | 5.75 | 20 | SH-MAL | 20 | 283 | Imm. |
| CP | 0.15 | 5.85 | 20 | SH-MAL | 20 | 5661 | Imm. |
| CP | 0.15 | 5.85 | 20 | SH-MAL | 20 | 566 | Imm. |
| CP | 0.15 | 5.85 | 20 | SH-MAL | 20 | 283 | Imm. |
| PB | 0.1 | 5.50 | 20 | SH-MAL | 20 | 3198 | ~30 |
| PB | 0.1 | 5.50 | 20 | SH-MAL | 20 | 566 | Imm. |
| PB | 0.1 | 5.50 | 20 | SH-MAL | 20 | 283 | Imm. |
| PB | 0.1 | 6.00 | 20 | SH-MAL | 20 | 3198 | Imm. |
| PB | 0.1 | 6.00 | 20 | HZ-AD | 20 | N/A | Did Not Gel |
| PB | 0.1 | 6.50 | 20 | SH-EP | 20 | N/A | <24 hrs |
| PB | 0.1 | 6.50 | 20 | SH-MAL | 20 | N/A | Imm. |
| PB | 0.1 | 6.50 | 40 | SH-MAL | 20 | N/A | Imm. |
| PB | 0.1 | 6.50 | 20 | SH-MAL | 20 | 5697 | Imm. |
| PB | 0.1 | 6.50 | 20 | SH-MAL | 20 | 5661 | Imm. |
| PB | 0.1 | 6.50 | 20 | SH-MAL | 20 | 3198 | Imm. |
| PB | 0.1 | 6.50 | 20 | SH-MAL | 20 | 2878 | Imm. |
| PB | 0.1 | 6.50 | 20 | SH-MAL | 20 | 2302 | Imm. |
| PB | 0.1 | 6.50 | 20 | SH-MAL | 20 | 1796 | Imm. |
| PB | 0.1 | 6.50 | 20 | SH-MAL | 20 | 1663 | Imm. |
| PB | 0.1 | 6.50 | 20 | SH-MAL | 20 | 1612 | Imm. |
| PB | 0.1 | 6.50 | 20 | SH-MAL | 20 | 1365 | Imm. |
| PB | 0.1 | 6.50 | 20 | SH-MAL | 20 | 1010 | Imm. |
| PB | 0.1 | 6.50 | 20 | SH-MAL | 20 | 566 | Imm. |
| PB | 0.1 | 6.50 | 20 | SH-MAL | 20 | 283 | Imm. |
| PB | 0.1 | 6.50 | 20 | SH-MAL | 15 | N/A | Imm. |
| PB | 0.1 | 6.50 | 20 | SH-MAL | 10 | N/A | Imm. |
| PB | 0.1 | 6.50 | 20 | SH-MAL | 5 | N/A | Imm. |
| PB | 0.1 | 6.50 | 20 | SH-MAL | 2.5 | N/A | Imm. |
| PB | 0.1 | 6.50(SH) + | 40 | SH-MAL | 15 | N/A | ~30 |
| | | 7.00 (MAL) | | (ONB) | | | |
| PB | 0.1 | 7.00 (SG) + | 20 | SG-NH | 20 | N/A | <24 hrs, |
| | | 9.00 (NH) | | | | | degraded in <3 |
| | | | | | | | days at 37° C. |
| PB | 0.1 | 7.00 (IC) + | 20 | IC-NH | 20 | N/A | Did Not Gel |
| | | 9.00 (NH) | | | | | |
| PB | 0.1 | 7.00 | 20 | SH-MAL | 2.5 | 400 | Imm. |
| PB | 0.1 | 7.00 | 20 | SH-MAL | 1 | 400 | Imm. |
| PB | 0.1 | 8.00 | 20 | SH-MAL | 2.5 | 400 | Imm. |
| PB | 0.1 | 8.00 | 20 | SH-MAL | 1 | 400 | Imm. |
| PB | 0.1 (+0.1/0.2M | 6.50 | 20 | SH-MAL | 20 | N/A | Imm. |
| | NaCl) | | | | | | |

In certain situations, the biomaterial/hydrogel/device/delivered product can be delivered by a delivery system in a fully formed state to a target location. Although a delivered product can be considered fully formed (i.e., the chemical reactions between the biomaterial components are completed), it can still undergo certain changes (e.g., in vivo changes) after delivery. For example, a delivered biomaterial product can continue to absorb water and/or swell and/or can expel salts, ions, and/or excipients. In some embodiments, a delivered biomaterial product can be a hydrogel that is formed by crosslinking of two or more biomaterial components. The term “hydrogel” can refer to any water-swollen (majority, >50%, of material mass is water), and cross-linked polymeric network produced by the reaction of one or more components (e.g., polymers, monomers) and/or a polymeric material that exhibits the ability to swell and retain a significant fraction of water within its structure, but will not dissolve in water.

In some embodiments, the biomaterial/hydrogel swells within the implantation space to lock or secure its placement. For example, a biomaterial in the form of a hydrogel may swell from about 1.25× to 10× its initial volume, such as about 1.5× to about 9×, 2× to 8×, 3× to 7×, 4× to 6×, or about 5× its initial volume. In some embodiments, the extruded biomaterial conforms to the space it is injected into. In some embodiments, the swelling of the biomaterial does not change volume within the implantation space, or shrinks to conform to a volume of the implantation space. In some embodiments, the apparatus injects a pre-formed biomaterial (does not cross-link, form, or gel in situ). Once injected, the biomaterial may or may not react with the implantation space. If a reaction does occur, it may be covalent or non-covalent. In some embodiments, the biomaterial adhesively interacts within the implantation space.

In one embodiment, the contrast-containing hydrogel may be stimulus-responsive, such that upon exposure to one or more stimuli, the hydrogel is reversed. The stimulus may cause the hydrogel to dissolve, degrade, de-precipitate, and/or liquefy. In one embodiment, the hydrogel is photoreversible, and the stimulus is light including ultraviolet (UV) or infrared (IR). In one embodiment, the light can be exposed above the skin and penetrate the skin such that the hydrogel is exposed, although infrared (IR) light is able to penetrate skin deeper than ultraviolet (IR). Photodegradation is most effective when the hydrogel is most superficial to the skin. Exposure to light can be accomplished with UV illumination using a UV laser, UV flashlamp, UV fluorescence microscope, or UV fiber optic. A light-emitting diode (LED), violet diode lasers, or a 2-photon light source can be used.

In one embodiment, the ultraviolet light that is used has a defined wavelength. Various wavelengths can impact the release of drugs from the hydrogel. The UV wavelengths can range from 260 nm to 405 nm, or any range in between.

The invention may be used for occlusion (for example, using any methods and/or compositions of the invention) of the femoral artery, popliteal artery, coronary and/or carotid artery; the esophagus, the oral cavity, nasopharyngeal cavity, car canal and tympanic cavity, sinuses of the brain, the arterial system, the venous system, heart, larynx, trachea, bronchi, stomach, duodenum, ileum, colon, rectum, bladder, kidney, ureter, ejaculatory duct, epididymis, vas deferens, the urethra, the uterine cavity, a vaginal canal, fallopian tubes, and cervix; any duct including a bile duct, a hepatic duct, a cystic duct, a pancreatic duct, or a parotid duct; an organ including a uterus, prostate, or any organ of the gastrointestinal tract or circulatory system or respiratory system or nervous system, or urological organ, or combinations thereof.

The following Examples are illustrative and should not be interpreted to limit the scope of the claimed subject matter.

### Example 1

Table 2 lists hydrogel formulations prepared for imaging. Macromer solutions were prepared in a CA-SC (citric acid-sodium citrate) buffer solution at a pH of 5.25. Formulations 1 and 2 were prepared using iohexol powder at 3 wt % and 10 wt %, respectively. At 10 wt %, iohexol was difficult to fully dissolve in the macromer solutions. Formulation 3 was prepared using gadodiamide powder. Formulation 4 was prepared using Omnipaque™ solution containing 300 mg/mL iohexol.

TABLE 2
Hydrogel formulations

| | | | | | Contrast |
|---|---|---|---|---|---|
| | | Macromer | | | Agent |
| Formulation | | Amount | | | Amount |
| No. | Macromer Type | (wt %) | Solvent | Contrast Agent | (wt %) |
| 1 | PEG-MAL | 20 | CA-SC (0.2M) | Iohexol | 3 |
| | (Macromer 1) | | | | |
| | PEG-SH | 20 | CA-SC (0.2M) | Iohexol | 3 |
| | (Macromer 2) | | | | |
| 2 | PEG-MAL | 20 | CA-SC (0.2M) | Iohexol | 10 |
| | (Macromer 1) | | | | |
| | PEG-SH | 20 | CA-SC (0.2M) | Iohexol | 10 |
| | (Macromer 2) | | | | |
| 3 | PEG-MAL | 20 | CA-SC (0.2M) | Gadodiamide | 5 |
| | (Macromer 1) | | | | |
| | PEG-SH | 20 | CA-SC (0.2M) | Gadodiamide | 5 |
| | (Macromer 2) | | | | |
| 4^{a} | PEG-MAL | 20 | CA-SC (0.2M) | Omnipaque ™ | — |
| | (Macromer 1) | | | | |
| | PEG-SH | 20 | CA-SC (0.2M) | Omnipaque ™ | — |
| | (Macromer 2) | | | | |

^{a}Macromers were dissolved in a 1:1 solution of Omnipaque ™ (solution containing 300 mg/mL iohexol) and CA-SC (0.2M) buffer solution.

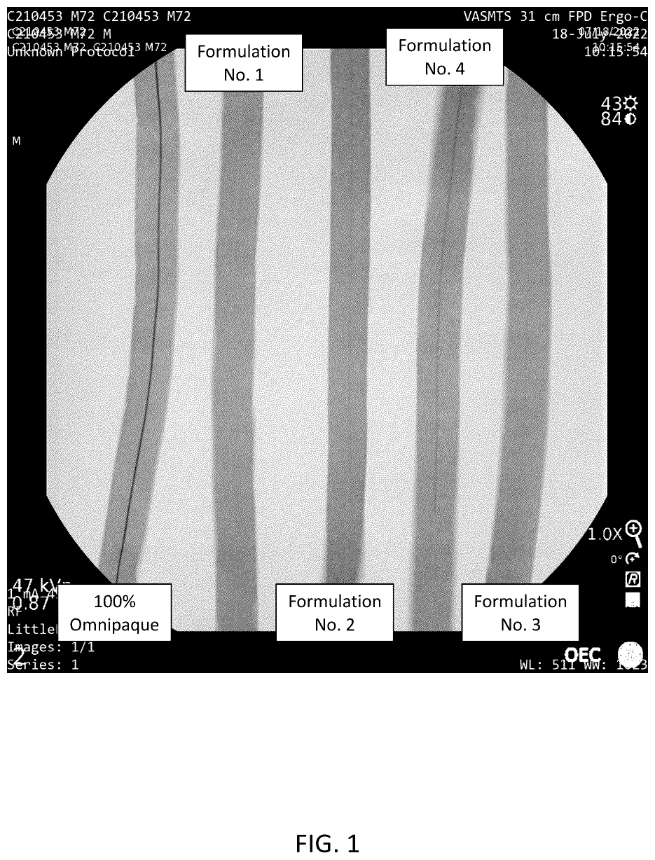

As shown in FIG. 1, formulations 1˜4 were implanted into synthetic vas mimics for visualization via fluoroscopy. A control was prepared using 100% Omnipaque™ solution. Formulations 1 and 3 provided implants that were the least radiopaque. Formulation 2, comprising 10 wt % iohexol, was more visible than formulation 1, as expected. Formulation 4 provided the best quality image.

### Example 2

Hydrogels, prepared according to embodiments of the present invention, were implanted into three male human cadavers (ages 35, 57, and 72 years old). The injections were visualized via fluoroscopy.

### Subject 1 (C210453-M72)—Male, 72 Years

The vasa of Subject 1 were cannulated using 22 G×1″ over-the-needle (OTN) catheters. Rather than cannulating through the vas wall, both vasa were ligated at the implantation site and catheters were inserted into the open vasa lumen. Correct placement of the catheters was verified using a guidewire. The right vas of this subject was injected with 0.25 mL of hydrogel formulation 4 (see Table 2) at 0.8 ml/min. The implant was observed to reach the distal vas past the inguinal canal. Following injection, implant material was observed leaking out of the vas when the catheter was removed, indicating that the full 0.25 mL volume was not delivered. The urogenital tract of this subject was excised, and the distal end of the implant was measured to be 22 cm from the implantation site.

The left vas was injected with 0.25 mL of Omnipaque™ at 0.8 mL/min. The 0.25 mL Omnipaque™ injection shows implant location and dimensions if a full 0.25 mL volume were to be implanted. This injection reached the seminal vesicle and ejaculatory duct.

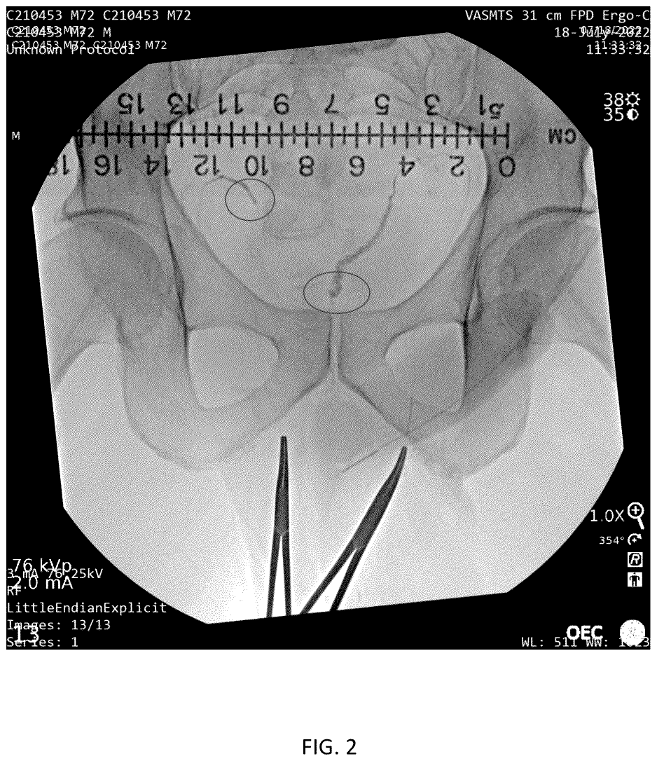

FIG. 2 is a fluoroscopy image showing the 0.25 mL injection of hydrogel formulation 4 in the right vas (left side of the image) and the 0.25 mL injection of 100% Omnipaque™ in the left vas (right side of the image). The distal ends of the injections are circled. The 0.25 mL distal end of the Omnipaque™ injection can be seen reaching the seminal vesicle and ejaculatory duct. The urogenital tract of this subject was excised, and the distal end of the right vas implant was measured to be 22 cm from the implantation site.

### Subject 2 (P210293-M57)—Male, 57 Years

The vasa of Subject 2 were cannulated using 22 G×1″ over-the-needle (OTN) catheters through the vasa walls. Correct placement of the catheters was verified using a guidewire. The right vas of this subject was injected with 0.25 mL of ADAM containing Omnipaque™ (see, e.g., formulation 4 (Table 2)) at 0.8 mL/min. The distal end of the injection was observed to be near the seminal vesicle and ejaculatory duct. The left vas of this subject was injected with 0.1 mL of ADAM containing Omnipaque™ (formulation 4) at 0.8 mL/min. The distal end of the injection was observed to be slightly entering the distal, tortuous portion of the vas.

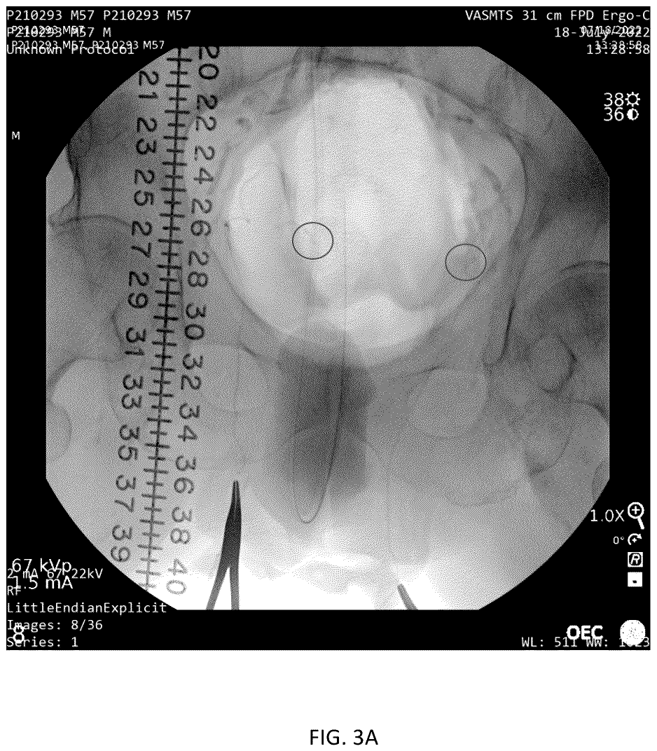

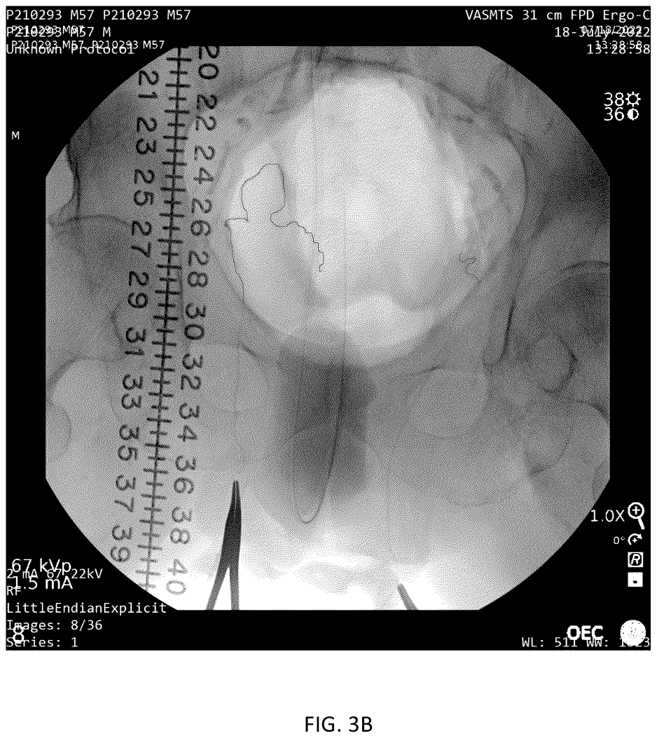

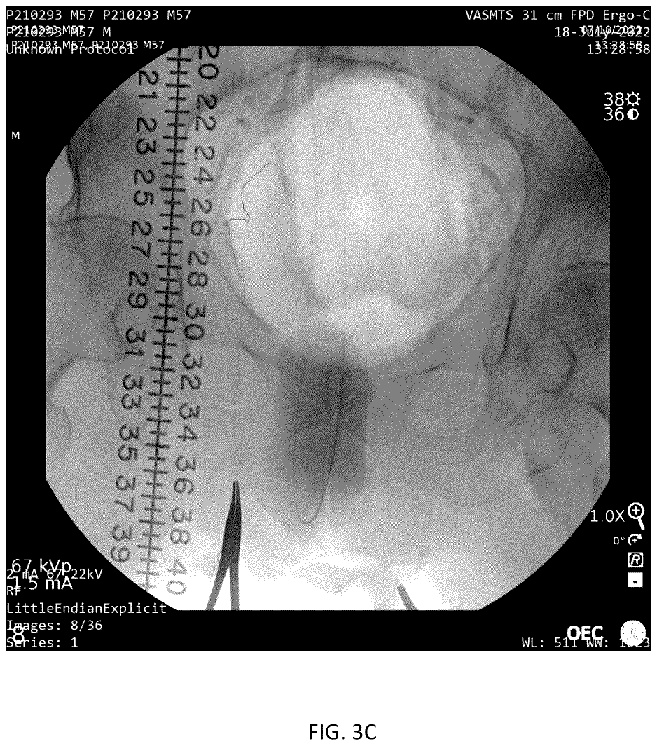

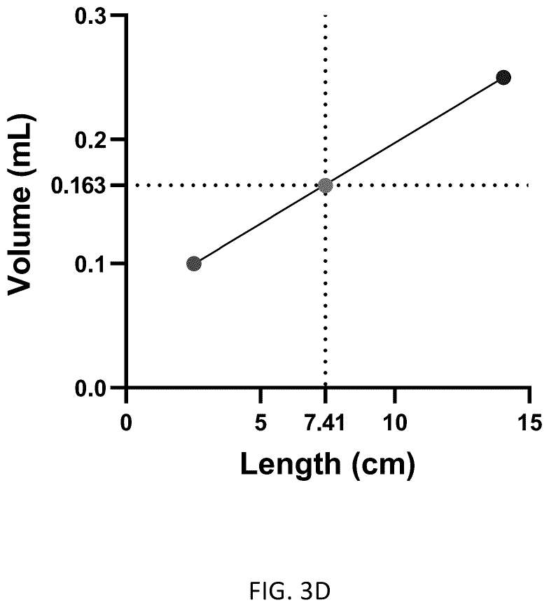

FIG. 3A is a fluoroscopy image showing the 0.25 mL injection of ADAM containing Omnipaque™ (formulation 4) in the right vas (left side of the image) and the 0.1 mL injection of ADAM containing Omnipaque™ (formulation 4) in the left vas (right side of the image). The distal ends of the injections are circled.

Subject 2 had a 0.25 mL implant that appeared to be in closest proximity to the seminal vesicle compared to the other subjects that received 0.25 mL implants. Since this would be the highest risk implant, this subject was selected for implant dimension analysis. Using FIJI, the implant lengths were measured from the inguinal canal to the distal end of the implant for both vasa. These lengths were correlated with the implant volumes (0.25 mL for the right vas and 0.1 mL for the left vas). Two points on a coordinate plane were selected using these corresponding lengths and volumes. An equation for a straight line that crosses these points was derived. Using this equation, the corresponding volume for what is suggested to be a possible safe maximum implant length was found.

As shown in FIG. 3B, lines were drawn starting from the inguinal canal, as the injection sites were either obscured or out of frame. In the right vas (left side), the 0.25 mL implant length shown by the line (inguinal canal to the distal end of the implant) was determined to be 14.04 cm. In the left vas (right side), the 0.1 mL implant length shown by the line (inguinal canal to the distal end of the implant) was determined to be 2.52 cm.

As shown in FIG. 3C, a line was drawn to represent a possible safe maximum implant length from the inguinal canal to the distal end of the implant. To mitigate the risk of the distal end of the implant being in close proximity to the seminal vesicle, an implant length that did not pass the apex at which the vas turns back to the prostate was selected. This length was determined to be 7.41 cm.

FIG. 3D Assuming x₁=2.52 cm, x₂=14.04 cm, y₁=0.1 mL, y₂=0.25 mL and given

$$y - {y}_{2} = \frac{{y}_{2} - {y}_{1}}{{x}_{2} - {x}_{1}} ( x - {x}_{2} ) ,$$

then >y=0.013×+0.067, where y is implant volume and x is implant length. Using this derived equation, a 7.41 cm implant would correspond to a 0.163 mL implant. Therefore, it is possible that a maximum safe implant dose be no greater than 163 μL. However, future additional cadaver implant length studies are required to provide more confidence in a maximum safe implant dose.

### Subject 3 (L220767-M35)—Male, 35 Years

The vasa of Subject 3 were cannulated using 22 G×1″ over-the-needle (OTN) catheters through the vasa walls. Correct placement of the catheters was verified using a guidewire. The right vas of this subject was injected with 0.1 mL of ADAM containing Omnipaque™ (formulation 4) at 0.8 mL/min. The left vas of this subject was injected with 0.25 mL of ADAM containing Omnipaque™ (formulation 4) at 0.8 mL/min.

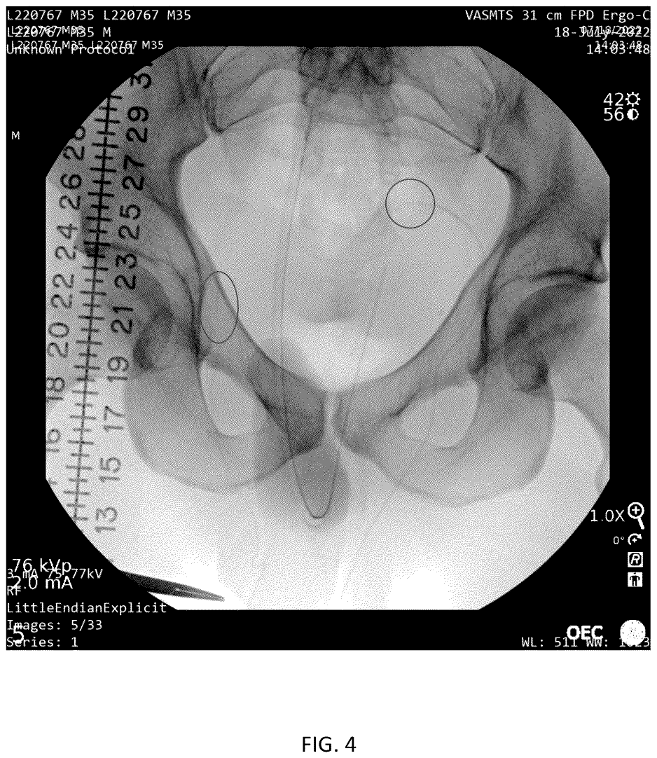

FIG. 4 is a fluoroscopy image showing the 0.1 mL injection of ADAM containing Omnipaque™ (formulation 4) in the right vas (left side of the image) and the 0.25 mL injection of ADAM containing Omnipaque™ (formulation 4) in the left vas (right side of the image). The distal ends of injections are circled. The 0.25 mL injection was observed to be more distal (deeper in vas) than the 0.1 mL injection.

### Example 3

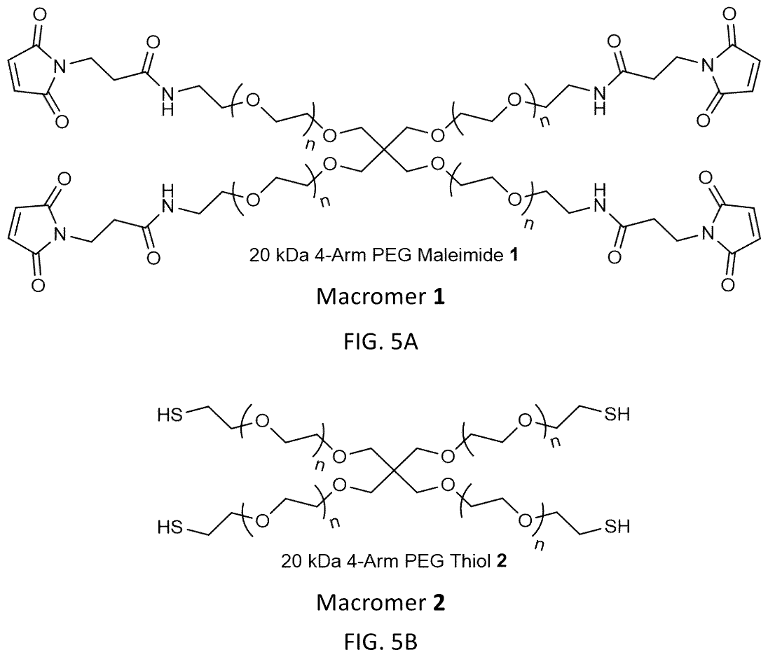

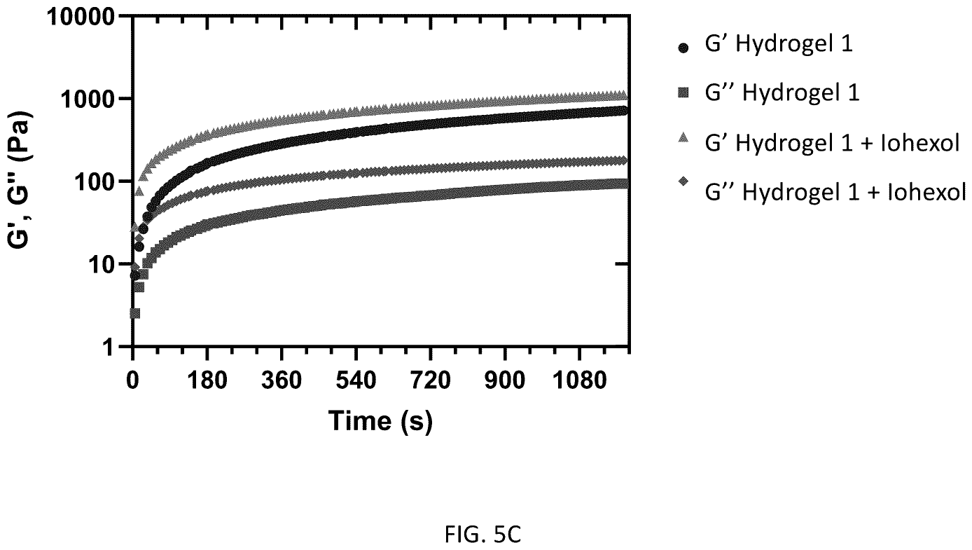

A hydrogel formulated according to an embodiment of the invention was prepared by mixing 200 mM citric acid-sodium citrate buffer solution at a pH of 5.25 with an equal amount of Omnipaque™ (solution containing 300 mg/mL iohexol). Macromers 1 and 2 (FIGS. 5A-B) were dissolved in the 1:1 buffer and Omnipaque™ solution at 20% w/w (see also Formulation 4 from Example 1). A control hydrogel was prepared without iohexol to compare gelation properties. Table 3 shows the formulation details for both hydrogels. Table 4 shows a comparison of the gelation properties of the hydrogel with iohexol to the control hydrogel.

TABLE 3
Formulation Specifications

| | Hydrogel 1 | | Hydrogel 1 + Iohexol | |
|---|---|---|---|---|
| pH | 5.15-5.35 | | 5.33 | |
| Citrate concentration | 200 | mM | 100 | mM |
| Macromer 1 Concentration | 20 | wt % | 20 | wt % |
| Macromer 2 Concentration | 20 | wt % | 20 | wt % |

| | | | |
|---|---|---|---|
| Iohexol (from Omnipaque ™) | none | 15 | wt % |

TABLE 4
Gelation Properties

| | Hydrogel 1 | | Hydrogel 1 + Iohexol | |
|---|---|---|---|---|
| Gelation Time | 12.2 | s | 12.1 | s |
| Storage Modulus (G′) @ 60 s | 57 | Pa | 185 | Pa |
| Storage Modulus (G′) @ 600 s | 428 | Pa | 747 | Pa |

| | | |
|---|---|---|
| Swelling Ratio (by weight) | 4.0x | 2.9x |

As shown in Table 4, while both hydrogels had similar gelation times, the hydrogel comprising iohexol showed increased stiffness and decreased swelling ratio. This is likely due to iohexol sequestration within the hydrogel network.

FIG. 5C shows the results of direct injection rheology testing. The hydrogel with iohexol gelled slightly faster and is slightly stiffer than the unmodified hydrogel at the time of extrusion (T₀). Both hydrogels are extremely weak (<200 Pa) upon injection.

The implants/compositions can be removed/reversed by any method applicable, including injecting a solution (such as into the vas deferens) to dislodge, de-precipitate, or dissolve the implant, or physically breaking apart the gel via vibration or electric stimulation. Reversal can be performed by way of degradation as a result of exposure to one or more stimuli such as light. In embodiments, the compositions comprise a polymer mass susceptible to on-command reversal in a body part upon exposure to one or more stimuli such that after the reversal is performed, the polymer mass no longer occludes the body lumen. Stimuli can facilitate disruption by way of one or more of ultrasound, x-ray, ultraviolet, visible, near infrared, or infrared light, thermal or wave energy, magnetic, electric, heat, vibrations, mechanical, or chemical disruption, aqueous solutions (neutral, basic, or acidic), organic solvent, aqueous-organic mixture, enzymatic, protein(s), peptide(s), small organic molecules, large organic molecules, nanoparticles, microparticles, quantum dots, carbon-based materials, and/or any combination thereof. Stimuli can also comprise providing a pressure gradient at both ends of the hydrogel, e.g., applying a positive pressure at the end of the hydrogel located upstream in a body part/lumen and applying a negative pressure at the downstream end to “push” or dislodge the implant/device out of the body part/lumen. A tool can be used by advancing a tool member at least partially into or through the implant/composition, such as a guidewire, a retrievable tool, an expandable member, a microcatheter, an ablation device or other tool member having a rigidity that is greater than the cohesion of the implant/composition. Exemplary tools are described in International Patent Application No. PCT/US2021/034562, incorporated by reference herein in its entirety. Once reversal is performed, the composition/device/implant disintegrates, de-precipitates, dislodges, or dissolves, allowing for the body part (such as a vas) to no longer be occluded.

### Example 4

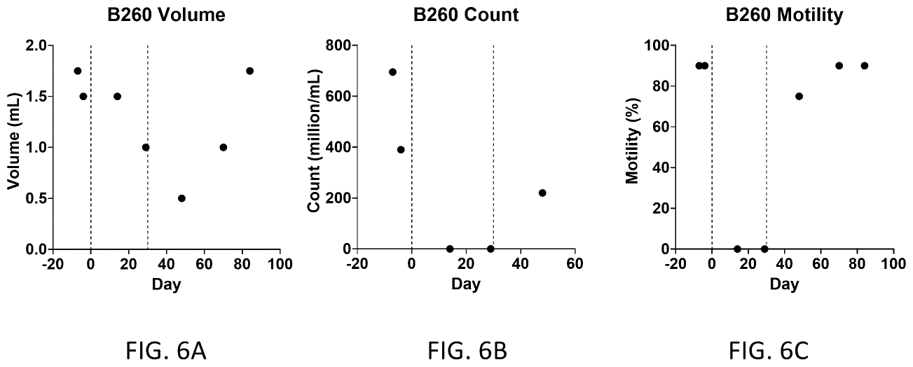

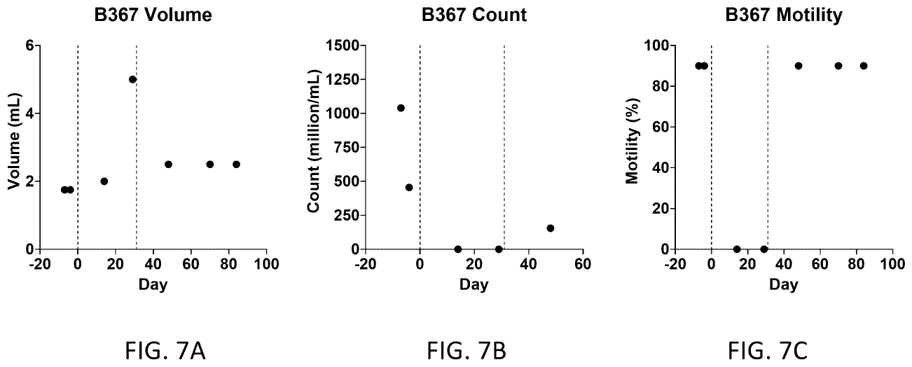

Reversal procedures were performed on two canine test subjects. Results from the mechanical reversal procedures are shown for the first canine subject (B260) in FIGS. 6A-C and for the second canine subject (B367) in FIGS. 7A-C. In embodiments, a device can be inserted into a body part (such as a vas deferens) upstream of the implant/composition and a force exerted on the implant/composition in a downstream direction (e.g., the direction from the testes towards the penile urethra) to cause at least a portion of the implant/composition to move from the vas deferens to the bladder. From the bladder, the portion of the implant/composition can exit the body via the urinary tract. In some embodiments, the removal device is an angioplasty device. Implantation of the composition is indicated by a first dashed line (day 0). Following implantation, azoospermia was found to occur in both subjects (FIGS. 6B and 7B). Upon reversal, as indicated by the second dashed line (day 30), the azoospermia was reversed. While the sperm count upon reversal was reduced in both cases, sperm motility returned to normal.

In embodiments, removal of the implant results in at least one of: A) a total sperm motility of sperm passing through the vas deferens at the implant location after the disruption of the implant being 30% to 100%, such as at least 30% to 70%, of a total sperm motility of the sperm passing through the vas deferens at a location upstream from the implant location, B) a total sperm concentration passing through the vas deferens at the implant location after the disruption of the implant being 30% to 100%, such as at least 30% to 70%, of a total sperm concentration passing through the vas deferens at the location upstream from the implant location, or C) an ejaculate volume passing through the vas deferens at the implant location after the disruption of the implant being 30% to 100%, such as at least 30% to 70%, of an ejaculate volume before passing through the vas deferens at the location upstream from the implant location.

The present invention has been described with reference to particular embodiments having various features. In light of the disclosure provided above and the claims provided below, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Any of the methods disclosed herein can be used with any of the compositions disclosed herein or with any other compositions. Likewise, any of the disclosed compositions can be used with any of the methods disclosed herein or with any other methods. Other embodiments of the invention will be apparent to those skilled in the art from consideration of the specification and practice of the invention.

It is noted in particular that where a range of values is provided in this specification, each value between the upper and lower limits of that range, to the tenth of the unit disclosed, is also specifically disclosed. Any smaller range within the ranges disclosed or that can be derived from other endpoints disclosed are also specifically disclosed themselves. The upper and lower limits of disclosed ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1-2. (canceled)

3. A composition comprising:
a first component comprising polyethylene glycol terminated with a first crosslinkable functional group; and
a second component comprising polyethylene glycol terminated with a second crosslinkable functional group;
wherein the first component and/or the second component further comprises one or more contrast agent; and
wherein the first component and the second component, upon mixing, are capable of forming a covalently cross-linked hydrogel as an end composition.

4. The composition of claim 3, wherein the end composition is a hydrogel comprising pores.

5. The composition of claim 4, wherein the pores have a diameter of no more than about 3 microns.

6. The composition of claim 5, wherein the contrast agent is iohexol.

7. (canceled)

8. The composition of claim 3, wherein:
the first crosslinkable functional group is a thiol; and
the second crosslinkable functional group is a maleimide.

9-11. (canceled)

12. The composition of claim 8, wherein the first component and the second component are present in the composition in equal amounts.

13. (canceled)

14. The composition of claim 8, wherein:
the first component and the second component are dissolved in a solvent; and
the solvent is a buffer comprising citric acid.

15-17. (canceled)

18. The composition of claim 14, wherein the first component and/or the second component has a molecular weight of up to about 50 kDa.

19. The composition of claim 18, wherein the polyethylene glycol of the first component and/or the second component comprises 4 arms.

20. The composition of claim 19, wherein the first component and the second component, upon mixing, are capable of forming the end composition at a gelation rate of less than 30 seconds.

21. The composition of claim 20, comprising a multi-arm polyethylene glycol terminated with a thiol and having a weight percent ranging from about 1 to 30% in the composition and/or a multi-arm polyethylene glycol terminated with a maleimide and having a weight percent ranging from about 1 to 30% in the composition.

22-25. (canceled)

26. A method of implanting a hydrogel composition, the method comprising:
identifying a target region within a body;
injecting a selected volume of a test composition into the target region;
visualizing the target region and the injected volume of the test composition using imaging;
removing the test composition from the target region;
selecting a desired length for an implanted hydrogel based on the visualizing;
determining an injection volume of a composition capable of forming the hydrogel that corresponds to the desired length for the implanted hydrogel; and
injecting the composition capable of forming the hydrogel into the target region.

27. The method of claim 26, wherein the target region is a vas deferens or fallopian tube.

28. The method of claim 27, wherein the imaging is fluoroscopy.

29. The method of claim 28, wherein the injection volume is less than about 200 microliters.

30. The method of claim 29, wherein the injection volume is less than about 175 microliters.

31. The method of claim 30, wherein the test composition comprises a contrast agent.

32. The method of claim 31, wherein:
the removing is performed by flushing the test composition from the target region with one or more flushing liquid; and
the flushing liquid comprises saline.

33. The method of claim 26, wherein the injecting of the composition capable of forming the hydrogel into the target region is performed in a manner to provide for temporary or permanent contraception.

34-37. (canceled)
